(19) United States
(12) Patent Application Publication
(10) Pub. No.: US 2024/0277279 A1
(43) Pub. Date: Aug. 22, 2024

(54) Sensing System for Sensing Biological Signals Externally on a Patient

(71) Applicant: BIOTRONIK SE & Co. KG, Berlin (DE)

(72) Inventors: Shayan GUHANIYOGI, Portland, OR (US); Matthew MELIUS, Milwauki, OR (US); Eric AUSTIN, Portland, OR (US); Hannes KRAETSCHMER, West Linn, OR (US); R. Hollis WHITTINGTON, Portland, OR (US); Dirk MUESSIG, West Linn, OR (US)

(73) Assignee: BIOTRONIK SE & Co. KG, Berlin (DE)

(21) Appl. No.: 18/681,160

(22) PCT Filed: Jul. 12, 2022
(86) PCT No.: PCT/EP2022/069405

§ 371 Date: Feb. 5, 2024

Related U.S. Application Data
(60) Provisional application No. 63/230,211, filed on Aug. 6, 2021.

(30) Foreign Application Priority Data
Sep. 7, 2021 (EP) 21195179.3

(51) Int. Cl. A61B 5/321; A61B 5/257; A61B 5/282; A61B 5/283

(52) U.S. Cl. CPC A61B 5/321; A61B 5/257; A61B 5/282; A61B 5/283

(57) ABSTRACT

A sensing system for sensing biological signals externally on a patient comprises a medical device comprising at least one electrode for sensing biological signals on a patient. An adapter element is mountable on said at least one electrode to electrically contact with the at least one electrode. A fastening component is mountable on said adapter element and configured to attach the medical device externally to the patient. A contact element is mountable to the fastening component and configured to attach externally to the patient's skin for receiving biological signals and conducting said biological signals to the adapter element. A casing serves for encasing the medical device and the adapter element in a mounted state in which the adapter element is mounted on said at least one electrode.

### CROSS-REFERENCE TO RELATED APPLICATIONS

This application is the United States National Phase under 35 U.S.C. § 371 of PCT International Patent Application No. PCT/EP2022/069405, filed on Jul. 12, 2022, which claims the benefit of European Patent Application No. 21195179.3, filed on Sep. 7, 2021 and U.S. Provisional Patent Application No. 63/230,211, filed on Aug. 6, 2021, the disclosures of which are hereby incorporated by reference herein in their entireties.

### TECHNICAL FIELD

The instant invention generally relates to a sensing system for sensing biological signals externally on a patient.

### BACKGROUND

Using a sensing system of the kind concerned herein biological signals shall be sensed outside of a patient. In particular, a sensing system of this kind may serve to sense electrocardiogram (ECG) signals, wherein for this contact elements, for example, in the shape of surface ECG electrodes may be attached to the patient's skin in order to make electrical contact to the patient, such that signals may be monitored on the patient.

Generally, different approaches exist to monitor biological signals, in particular cardiac signals such as electrocardiogram signals, on a patient.

If a short-term monitoring is required, a monitoring device may be attached externally to a patient in order to sense biological signals on the patient, for example over a duration of multiple days or a few weeks.

If a long-term monitoring shall be conducted, an implantable medical device in the shape of a biological sensor may be implanted in a patient, in particular subcutaneously within a minimally invasive procedure. An implantable medical device of this kind may remain within the patient over a rather long duration, for example several months or even years, such that signals may be sensed in the context of a long-term monitoring, for example to detect cardiac arrhythmias or the like.

There is a general desire to provide for a short-term sensing system which may be easily applied to a patient and may be comfortably worn externally by the patient.

The present disclosure is directed toward overcoming one or more of the above-mentioned problems, though not necessarily limited to embodiments that do.

### SUMMARY

It is an object of the instant invention to provide a sensing system and a method for providing a sensing system which allow, in an easy and cost-efficient manner, for a sensing of biological signals externally on a patient, while achieving a reliable monitoring as well as an increased comfort for a patient.

In one aspect, a sensing system for sensing biological signals externally on a patient comprises: a medical device comprising at least one electrode for sensing biological signals on a patient: an adapter element mountable on said at least one electrode to electrically contact with the at least one electrode; a fastening component mounted or mountable on said adapter element and configured to attach the medical device externally to the patient or patient's body: a contact element mounted or mountable to the fastening component and configured to attach externally to the patient's skin for receiving biological signals and conducting said biological signals to the adapter element; and a casing for encasing the medical device and the adapter element in a mounted state in which the adapter element is mounted on said at least one electrode.

The fastening component may comprise a contact face configured to touch or contact the patient's skin. The contact element is preferably attachable/mountable or configured to be attached/mounted to the contact face of the fastening component.

In order to be able to transmit or conduct the biological signals from the contact element to the at least one electrode, the adapter element, the fastening component and/or the contact element may be electrically conductive or at least partially made of an electrically conductive material.

The medical device in particular may be an implantable medical device which generally is designed for implantation into a patient. Within the implantable medical device, the at least one electrode may be configured to electrically contact biological tissue to sense biological signals in an implanted state of the medical device.

For example, the medical device may be a biosensor which originally is designed for subcutaneous implantation into a patient for sensing electrocardiogram (ECG) signals.

Within the sensing system, a medical device—which may originally be designed for a different purpose, in particular for implantation into a patient—may be used for an external sensing of biological signals, in particular cardiac signals, such as electrocardiogram signals. In particular, the medical device may be an implantable loop recorder or implantable heart monitor. For this, the medical device is combined with one or multiple adapter elements, each of which contains or is connected to a fastening component. The fastening component preferably fastens to contact elements that are attached externally on the patient's skin in order to pick up biological signals on the patient and to conduct the biological signals via the fastening component of the adapter element to the at least one electrode of the medical device.

It generally can be assumed that an implantable sensor device comprises a processing circuitry which makes the device usable also for an external monitoring. Hence, by means of the instant sensing system it is proposed to use the medical device, which originally may not have been designed for an external use, for an external monitoring by adapting the medical device for an external use using one or multiple adapter elements and one or multiple contact elements.

Within an implantable medical device the at least one electrode is designed to electrically contact with surrounding tissue when the implantable medical device is implanted in a patient. However, an electrode of this kind may, as such, not be suitable when using the implantable medical device externally on a patient. Therefore, an adapter element is used to engage with the at least one electrode of the medical device in order to make electrical contact to the at least one electrode, wherein an attachment on the patient is achieved by means of the fastening component and/or the contact element. At least the fastening component is in contact with the adapter element and hence may pick up biological signals from the patient's skin and/or the contact element for conducting the biological signals via the adapter element towards the at least one electrode. It shall be noted that the contact element is not necessarily a part of the system. For example, the fastening component may be attached directly to the patient's skin and made of an electrically conductive material. Thus, the system could function equally or at least similarly even without the contact element. However, the system could function also equally or at least similarly without the fastening component, wherein the adapter element is connected/connectable directly to the contact element. Alternatively, the fastening component may be a part of the adapter element.

The contact element may have the shape of a commercially available ECG electrode which may be adhesively attached to a patient.

The fastening component and/or the contact element, in one embodiment, is releasably connectable to the adapter element. For example, the fastening component and/or the adapter element may comprise a button connector which allows for an attachment of the fastening component and/or the adapter element to the contact element, wherein the fastening component and/or the adapter element may be attachable to different contact elements, in particular contact elements of different size.

The adapter element and/or the fastening component is/are electrically conductive such that biological signals may be conducted from the contact element towards the at least one electrode of the medical device for processing the biological signals within the medical device.

The assembly of the adapter element and the medical device is encased in a casing, which preferably is made from a biocompatible material. The medical device with the adapter element mounted thereon hence is received within a protective casing, which is placed externally on a patient and protects the medical device with the adapter element arranged thereon towards the outside.

In one embodiment, at least one of the medical device and the adapter element (preferably including the fastening component) are removable from the casing. This allows for a reuse of the casing as well as the medical device and the adapter element received therein. For example, by removing the medical device and the adapter element from the casing, the medical device as well as the adapter element and the casing may be thoroughly cleaned, such that the casing, the medical device and the adapter element may be reused on another patient. Also, whereas the medical device and/or the adapter element may be designed for a reuse, the casing may be designed only for a one-time usage, in which case the casing may be discarded after use and after removal of the medical device and the adapter element received therein.

In one embodiment, the adapter element is releasably mounted on the at least one electrode. In particular, the mounting of the adapter element on the medical device may be such that no modification to the medical device is required. By hence placing the adapter element on the medical device, an electrical contact in between the adapter element and the at least one electrode of the medical device can be established, wherein after use the adapter element may be again removed from the medical device in order to, for example, prepare the medical device and/or the adapter element for a reuse, for example by cleaning the medical device and the adapter element.

In one embodiment, the adapter element may be fixed on the medical device, for example, using a screw or the like.

In an embodiment in which the adapter element is to be non-removably connected to the medical device and/or the at least one electrode, for example a welding connection in between the adapter element and the medical device may be established.

In one embodiment, the adapter element comprises a body forming a reception opening for receiving the at least one electrode therein. By inserting the medical device with an electrode arranged thereon into the reception opening of the body of the adapter element an electrical contact in between the adapter element and the electrode may be established. This allows for an easy mounting of the adapter element on the medical device, and in addition a reliable operation, in particular a reliable electrical contact in between the adapter element and the electrode of the medical device.

In one embodiment, the medical device comprises a housing forming a main housing portion and a header portion extending from the main housing portion. An electronics module may, for example, be arranged in or on the main housing portion, whereas, for example, one or multiple electrodes are arranged in or on the header portion. The header portion may, for example, extend linearly from the main housing portion, wherein the header portion may be flexibly deformable, or may have a rigid structure such that it is not flexibly deformable.

Herein, a first electrode may, for example, be arranged on an end of the header portion which is distal with respect to the main housing portion. Another, second electrode may, in one embodiment, be placed on the main housing portion, for example at an end of the main housing portion opposite to the header portion. Each electrode herein may be associated with a dedicated adapter element, such that the first electrode is electrically in contact with a first adapter element connected to a first contact element and/or a first fastening component, and the second electrode is electrically in contact with another, second adapter element connected to a second contact element and/or a second fastening component, such that each electrode is attached to the patient for sensing biological signals on the patient.

In one embodiment, the adapter element and the fastening element are formed in one piece. This simplifies the structure and assembly of the system.

In another embodiment, the adapter element, the fastening element and the contact element are formed in one piece. This particularly simplifies the structure and assembly of the system.

Additionally or alternatively, at least two of the adapter element, the fastening element and the contact element may be connectable to each other via button connectors.

In another aspect, a method for providing a sensing system for sensing biological signals externally on a patient comprises: providing an implantable medical device comprising at least one electrode for sensing biological signals on a patient: mounting an adapter element on said at least one electrode to electrically contact with the at least one electrode; providing a fastening component mounted or mountable on said adapter element (3A, 3B) and configured to attach the medical device (1) externally to the patient: providing a contact element mounted or mountable to the fastening component and configured to attach externally to the patient's skin for receiving biological signals and conducting said biological signals to the adapter element; and encasing, using a casing, the medical device and the adapter element in a mounted state in which the adapter element is mounted on said at least one electrode.

In yet another aspect, a sensing system is used for sensing biological signals externally on a patient, the sensing system comprising: an implantable medical device comprising at least one electrode for sensing biological signals on a patient: an adapter element mountable on said at least one electrode to electrically contact with the at least one electrode: a fastening component (4) mounted or mountable on said adapter element (3A, 3B) and configured to attach the medical device (1) externally to the patient: a contact element mounted or mountable to the fastening component and configured to attach externally to the patient's skin for receiving biological signals and conducting said biological signals to the adapter element; and a casing for encasing the medical device and the adapter element in a mounted state in which the adapter element is mounted on said at least one electrode.

The advantages and advantageous embodiments described above for the sensing system equally apply also to the method and the use, such that it shall be referred to the above in this respect.

By using an implantable medical device in combination with one or multiple adapter elements and one or multiple contact elements for electrically attaching one or multiple electrodes of the implantable medical device externally to the patient's skin, an implantable device which as such is designed for an implanted use may be used as an external monitoring device. By encasing the arrangement of the medical device and one or multiple adapter elements attached to the medical device a functional module is provided which may be placed outside on a patient and may be slender in build.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The various features and advantages of the present invention may be more readily under-stood with reference to the following detailed description and the embodiments shown in the drawings. Herein,

FIG. 1 shows a schematic drawing of an implantable medical device implanted in a patient;

FIG. 2 shows a schematic view of an embodiment of an implantable medical device together with an external communication device:

FIG. 3 shows a view of an implantable medical device comprising a first electrode on a header portion and a second electrode on a main body portion:

FIG. 4 shows an adapter element to be mounted on an electrode of the medical device, including the fastening component, in combination with a contact element to be attached to a patient:

FIG. 5 shows the medical device of FIG. 3 together with two adapter elements (including fastening component) for attaching the medical device to a patient:

FIG. 6 shows the arrangement of FIG. 5 encased in a casing; and

FIG. 7 shows the arrangement of FIG. 6 in a semitransparent view of the casing.

### DETAILED DESCRIPTION

Subsequently, embodiments of the present invention shall be described in detail with reference to the drawings. In the drawings, like reference numerals designate like structural elements.

It is to be noted that the embodiments are not limiting for the present invention, but merely represent illustrative examples.

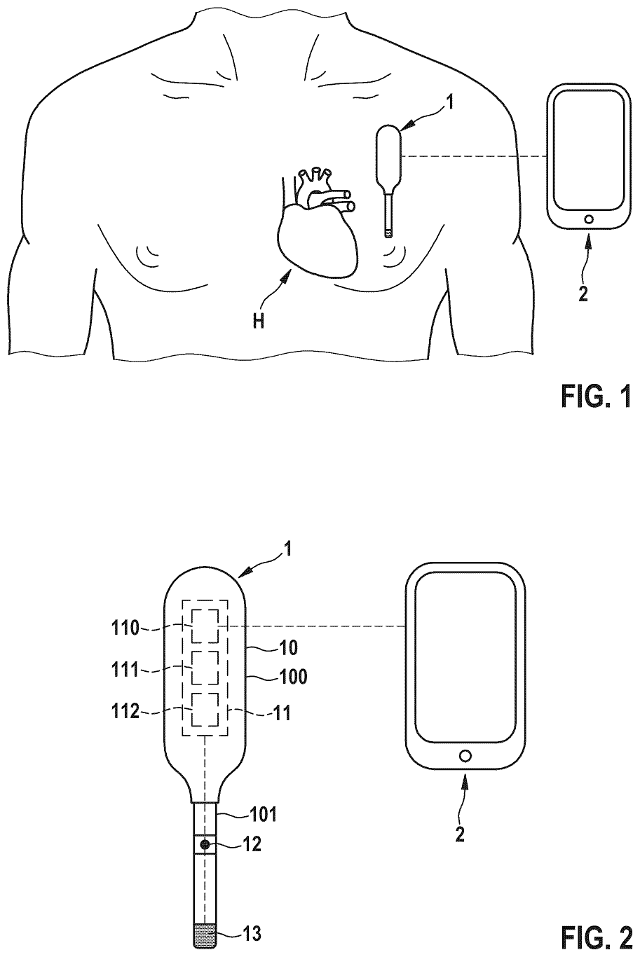

Within the instant text it is proposed to use an implantable medical device 1, as shown in one embodiment in FIGS. 1 and 2, externally on a patient to provide for a sensing system for an external monitoring and sensing of biological signals.

Referring to FIG. 1, in one embodiment an implantable medical device 1 is designed for implantation (for example, subcutaneous implantation) into a patient. The implantable medical device 1 may, for example, be a sensor device for measuring biological signals and may be designed to be implanted subcutaneously within a patient in proximity to the patient's heart H, for example left-pectorally, in order to sense biological signals from the patient's heart H, such as electrocardiogram (ECG) signals.

In an implanted state, the implantable medical device 1, e.g., shall sense signals and shall transfer information relating to sensed signals to an external communication device 2, for example within a home monitoring system. For this, the implantable medical device 1 may employ a standardized communication technology, such as the Zigbee technology or the Bluetooth Low Energy (BLE) technology.

Referring now to FIG. 2, in one embodiment the implantable medical device 1 comprises a housing 10 forming a main housing portion 100 and a header portion 101 linearly extending from the main housing portion 100. An electronics module 11 is arranged within the main housing portion 100, the electronics module 11 forming a communication circuitry 110 for establishing a communication with an external communication device 2. Further, the electronics module 11 comprises a processing circuitry 112 for processing signals for stimulation or sensing, and an energy storage 111 for supplying energy in the shape of a battery for operating the implantable medical device 1.

The header portion 101, in the embodiment of FIG. 2, comprises an electrode arrangement 13 comprising an electrode which is arranged at a distal end of the header portion 101 and hence at a remote location with respect to the electronics module 11 arranged within the main housing portion 100. In addition, an antenna 12 is formed on the header portion 101, the antenna 12 serving to transmit and/or receive signals, in cooperation with the communication circuitry 110, for communication with the external communication device 2.

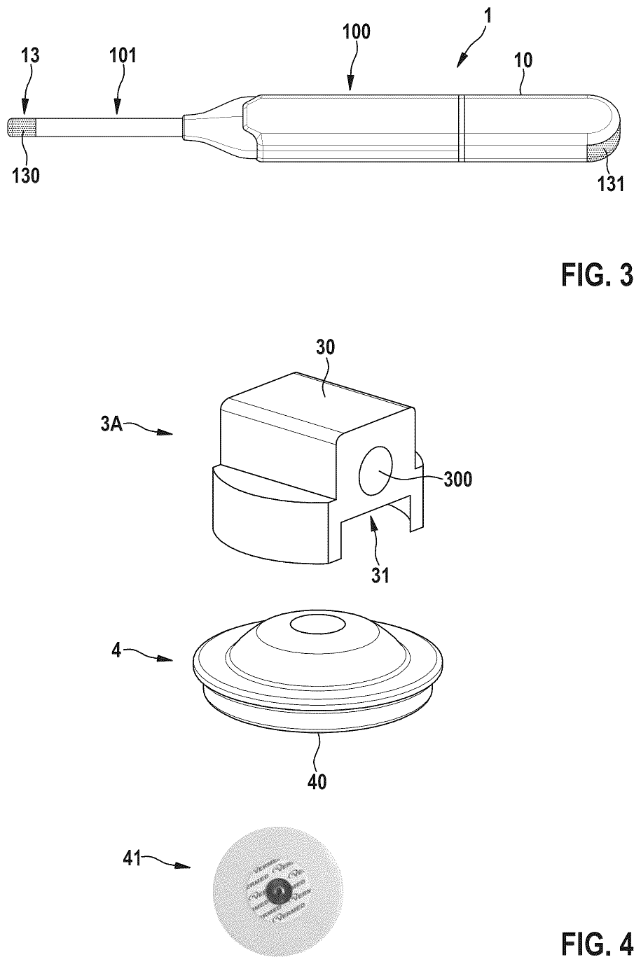

Referring now to FIGS. 3 to 5, in one embodiment the implantable medical device 1 comprises two electrodes 130, 131 with a first electrode 130 and a second electrode 131 together forming an electrode arrangement 13 for sensing biological signals, in particular electrocardiogram signals. The electrodes 130, 131 herein are designed particularly for sensing signals in an implanted state of the implantable medical device 1 by making electrical contact to tissue surrounding the implantable medical device 1.

As it is visible from FIG. 3, a medical device 1 designed for implantation generally is slender in built and comprises a minimized form factor. A medical device 1, for example in the shape of a biosensor for sensing electrocardiogram signals, generally comprises circuitry which however makes the medical device 1 suitable also for an external use to sense biological signals, such as electrocardiogram signals, externally on a patient.

It hence, in the context of this text, is proposed to use a medical device 1 which originally is designed for a different purpose, such as for implantation, for an external sensing by adapting the medical device 1 for an external use.

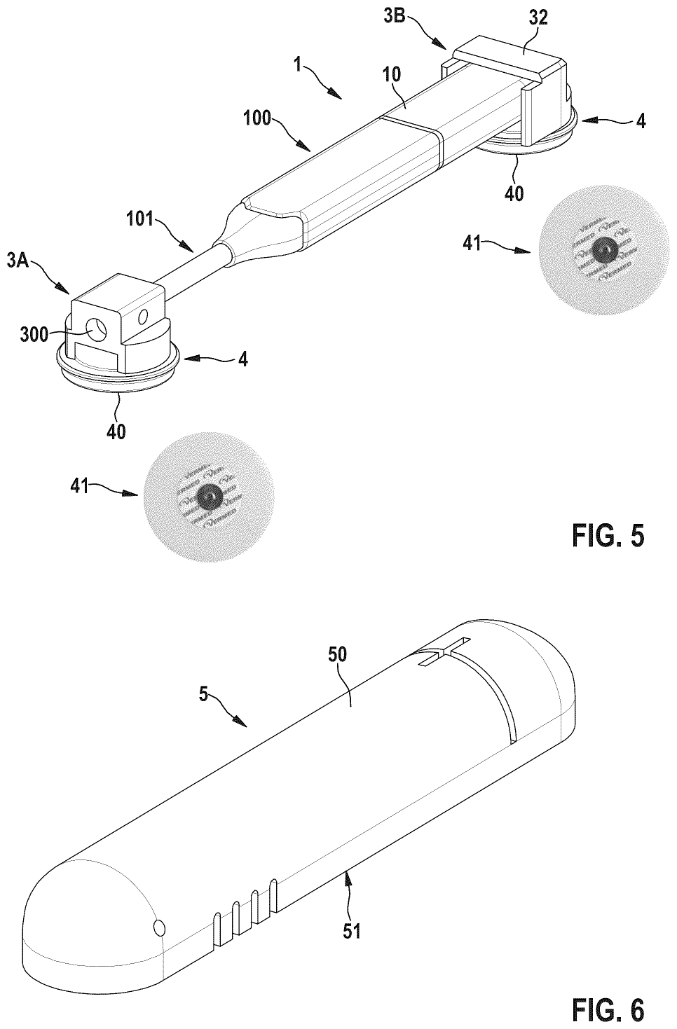

For this, adapter elements 3A, 3B are provided, as shown in FIGS. 4 and 5, which are associated with the electrodes 130, 131 of the medical device 1 and may be mounted on the medical device 1 such that the adapter elements 3A, 3B make electrical contact with the electrodes 130, 131.

For example, an adapter element 3A adapted to contact the electrode 130 on the header portion 101 of the housing 10 of the medical device 1 may comprise a body 30 forming a reception opening 300 in the shape of a through-hole into which the header portion 101 may be inserted in order to bring the electrode 130 arranged distally on the header portion 101 into electrical contact with the body 30 of the adapter element 3A.

An adapter element 3B associated with the electrode 131 arranged on the main housing portion 100, in turn, may comprise a box-like body 32 in which the main housing portion 100 with an end remote from the header portion 101 may be received, such that the electrode 131 electrically contacts with the body 32.

Each of the adapter elements 3A, 3B herein includes or is connectable to a fastening component 4 in the shape of, for example, a button connector. The fastening component enables releasably connecting the adapter elements 3A, 3B to contact elements 41, by means of contact faces 40, such that the contact elements 41 electrically contact with the patient and in addition are in electrical contact with the adapter elements 3A, 3B via the fastening components 4, and, via the adapter elements 3A, 3B, with the electrodes 130, 131 of the electrode arrangement 13 of the medical device 1. The contact elements 41 may be, for example, commercially available surface ECG electrodes which may be adhesively attached to the patient's skin. Each of the adapter elements 3A, 3B may, for example, comprise a fastening device 31, for example, in the shape of a button connector, such that the adapter elements 3A, 3B may be releasably connected to the contact elements 41 and/or to the fastening components 4.

The contact elements 41 (e.g., by means of a contact face 40) may be placed on the patient's skin and may be adhesively attached to the patient, such that the contact elements 41 electrically contact with the patient and in addition are in electrical contact with the adapter elements 3A, 3B and/or fastening components 4 and, via the adapter elements 3A, 3B, with the electrodes 130, 131 of the electrode arrangement 13 of the medical device 1.

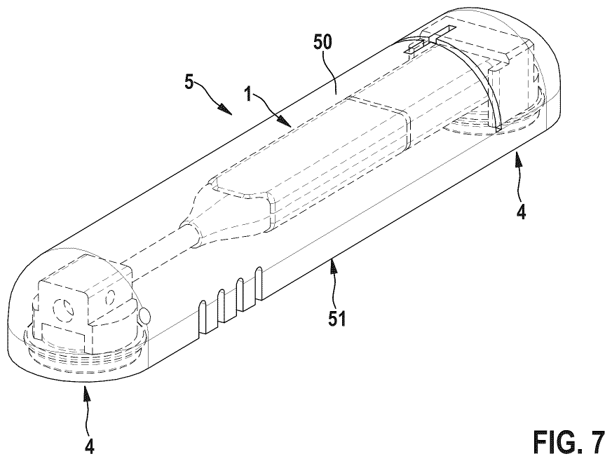

Referring now to FIGS. 6 and 7, the assembly of FIG. 5 may be received within a casing 5 in order to provide for a modular device which may be placed and used on a patient. The casing 5 herein comprises an outer housing wall 50 which provides for a protective shielding towards the outside. The casing 5 may also be called external casing 5 or patch 5. By means of a bottom face 51 the casing 5 may be placed on a patient, wherein the fastening components 4 of the adapter elements 3A, 3B, are accessible at the bottom face 51 and hence may be brought into contact with contact elements 41 on the patient's skin by placing the casing 5 on the patient and/or the contact elements 41.

The casing 5 may, for example, be made from a biocompatible material. In that the casing 5 is adapted to the slender shape of the medical device 1, the sensing system as provided by the arrangement of FIGS. 6 and 7 is slender in built and hence comfortable to wear for a patient.

The casing 5 may comprise an opening, or may be adapted for disassembly, for removing the medical device 1 and the adapter elements 3A, 3B, for example including the fastening components 4, from the casing 5. In addition, the adapter elements 3A, 3B may be removable from the medical device 1, such that the sensing system may fully be disassembled. This allows for a reuse of the casing 5, the medical device 1 and/or the adapter elements 3A, 3B, wherein for a reuse the components of the system may be disassembled and thoroughly cleaned and may subsequently be reused on a different patient.

In one embodiment, the medical device 1, the adapter elements 3A, 3B (e.g., including fastening components 4) and the casing 5 are reusable.

In another embodiment, only the medical device 1 and the adapter elements 3A, 3B (e.g., including fastening components 4) are reusable, whereas the casing 5 is to be disposed after a one-time use.

The implantable medical device 1 may be usable within the sensing system in a complete production state in which it also is usable as an implantable medical device for implantation into a patient.

In another embodiment, the medical device 1 is not in a complete production state when used in the sensing system for external sensing, but, for example, only components, such as an electrode arrangement 13 and an electronics module 11, e.g., on a circuit board, are used for the sensing system and for this are encased in a casing 5.

The contact elements 41 and/or the fastening components 4 are removable from the adapter elements 3A, 3B, wherein the contact elements 41 and/or the fastening components 4 of arbitrary shape may be selected for use in the sensing system. The contact elements 41 and or the fastening components 4 may, for example, be or compatible with commercially available surface ECG electrodes.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 (Implantable) medical device
10 Housing
100 Main housing portion
101 Header portion
11 Electronics module
110 Communication circuitry
111 Energy storage
112 Processing circuitry
12 Antenna
13 Electrode arrangement
130, 131 Electrode
2 External communication device
3A, 3B Adapter element
30 Body (of adapter element 3A)
300 Reception opening
31 Fastening device
32 Body (of adapter element 3B)
4 Fastening component
40 Contact face
41 Contact element
5 Casing
50 Housing wall
51 Bottom face
H Heart

1. A sensing system for sensing biological signals externally on a patient, comprising:
a medical device comprising at least one electrode for sensing biological signals on a patient;
an adapter element mountable on said at least one electrode to electrically contact with the at least one electrode;
a fastening component mountable on said adapter element and configured to attach the medical device externally to the patient;
a contact element mountable to the fastening component and configured to attach externally to the patient's skin for receiving biological signals and conducting said biological signals to the adapter element; and
a casing for encasing the medical device and the adapter element in a mounted state in which the adapter element is mounted on said at least one electrode.

2. The sensing system according to claim 1, wherein the medical device is implantable into a patient, said at least one electrode being configured to electrically contact biological tissue to sense biological signals in an implanted state of the medical device.

3. The sensing system according to claim 1, wherein the medical device is a bio-sensor for sensing electrocardiogram signals.

4. The sensing system according to claim 1, wherein the fastening component is releasably connectable to said adapter element.

5. The sensing system according to claim 1, wherein at least one of the medical device and the adapter element are removable from the casing.

6. The sensing system according to claim 1, wherein the adapter element is releasably mountable on said at least one electrode.

7. The sensing system according to claim 1, wherein the adapter element comprises a body forming a reception opening for receiving said at least one electrode therein.

8. The sensing system according to claim 1, wherein the medical device comprises a housing forming a main housing portion and a header portion extending from the main housing portion, and an electronics module being arranged in or on the main housing portion.

9. The sensing system according to claim 8, wherein the header portion-linearly extends from the main housing portion.

10. The sensing system according to claim 8, wherein a first electrode is placed on the header portion and a second electrode is placed on the main housing portion.

11. The sensing system according to claim 10, wherein an adapter element is mountable on each of the first electrode and the second electrode.

12. The sensing system according to claim 1, wherein the adapter element and the fastening element are formed in one piece.

13. The sensing system according to claim 1, wherein the adapter element, the fastening element and the contact element are formed in one piece.

14. A method for providing a sensing system for sensing biological signals externally on a patient, comprising:
providing an implantable medical device comprising at least one electrode for sensing biological signals on a patient;
mounting an adapter element on said at least one electrode to electrically contact with the at least one electrode;
providing a fastening component mountable on said adapter element and configured to attach the medical device externally to the patient;
providing a contact element mountable to the fastening component and configured to attach externally to the patient's skin for receiving biological signals and conducting said biological signals to the adapter element; and
encasing, using a casing, the medical device and the adapter element in a mounted state in which the adapter element is mounted on said at least one electrode.

15. A use of a sensing system for sensing biological signals externally on a patient, the sensing system comprising:
an implantable medical device comprising at least one electrode for sensing biological signals on a patient;
an adapter element mountable on said at least one electrode to electrically contact with the at least one electrode;
a fastening component mountable on said adapter element and configured to attach the medical device externally to the patient;
a contact element mountable to the fastening component and configured to attach externally to the patient's skin for receiving biological signals and conducting said biological signals to the adapter element; and
a casing for encasing the medical device and the adapter element in a mounted state in which the adapter element is mounted on said at least one electrode.
